SEQUENCE LISTING (12 sequences)

SEQ ID NO: 1 - protein, 7 aa, Artificial Sequence
  Synthetic Construct
LRRASLG

SEQ ID NO: 2 - DNA, 33 bp, Artificial Sequence
  Synthetic Construct
cggaattcatatggtgcccatccaaaaagtcca

SEQ ID NO: 3 - DNA, 48 bp, Artificial Sequence
  Synthetic Construct
gcggatccttagcccaggctcgcacgacgcaggcacccagggctgagg

SEQ ID NO: 4 - DNA, 53 bp, Artificial Sequence
  Synthetic Construct
gcggatccttagcccaggctcgcgcggcgcagggggcccaggctcgcacgacg

SEQ ID NO: 5 - DNA, 53 bp, Artificial Sequence; identical to SEQ ID NO: 4
  Synthetic Construct

SEQ ID NO: 6 - DNA, 47 bp, Artificial Sequence
  Synthetic Construct
catgccatgggcatcaccatcatcaccatgatattcaaaaaagagtg

SEQ ID NO: 7 - DNA, 50 bp, Artificial Sequence
  Synthetic Construct
gctctagattagcccaggctcgcacgacgcaggatggaggtacggcggta

SEQ ID NO: 8 - protein, 11 aa, Artificial Sequence
  Synthetic Construct
EAIYAAPFAKK

SEQ ID NO: 9 - DNA, 33 bp, Artificial Sequence; identical to SEQ ID NO: 2
  Synthetic Construct

SEQ ID NO: 10 - DNA, 67 bp, Artificial Sequence
  Synthetic Construct
cgggatcctcattatttttttttcgcaaacggcgccgcatagatcgcttcgcacccagggctgaggt

SEQ ID NO: 11 - DNA, 61 bp, Artificial Sequence
  Synthetic Construct
cgggatcctttttttttcgcaaacggcgccgcatagatcgcttcgcacccagggctgaggt

SEQ ID NO: 12 - DNA, 71 bp, Artificial Sequence
  Synthetic Construct
cgggatcctcattatttttttttcgcaaacggcgccgcatagatcgcgggtttttttttcgcaaacggcgc

(19) United States
(12) Patent Application Publication
(10) Pub. No.: US 2006/0105407 A1
(43) Pub. Date: May 18, 2006

(54) Protein chip for analyzing interaction between protein and substrate peptide thereof

(76) Inventors: Sang Yup Lee, Daejeon (KR); Seok Jae Lee, Daejeon (KR)

Correspondence Address: INTELLECTUAL PROPERTY / TECHNOLOGY LAW, PO BOX 14329, RESEARCH TRIANGLE PARK, NC, 27709, US

(21) Appl. No.: 10/541,261

(22) PCT Filed: Oct. 18, 2003
(86) PCT No.: PCT/KR03/02183

§ 371 Date: Sep. 26, 2005

(30) Foreign Application Priority Data
Jan. 4, 2003 (KR) 10-203-0000464

(51) Int. Cl. G01N 33/537; C12M 1/34

(52) U.S. Cl. 435/7.92; 435/287.2

(57) ABSTRACT

A protein chip of a S—L—SP form wherein a substrate peptide (SP) is immobilized on a solid substrate (S) by the mediation of a linker protein (L). Such chip is usefully employed in a method for analyzing the interaction between a reactive protein and its substrate peptide involving the steps of: adding to the protein chip a reactive protein showing a specific interaction with the substrate protein immobilized on the protein chip; and detecting the interaction between the reactive protein and the substrate peptide. The use of such protein chip and methodology allows an increase in the reactivity between a peptide with low molecular weight and an enzyme with high molecular weight and between the peptide and a reactive antibody on the protein chip, so that the interaction between the peptide and the protein can be analyzed rapidly and massively.

### TECHNICAL FIELD

The present invention relates to a protein chip for analyzing the interaction between a protein and its substrate peptide. More particularly, the present invention relates to a protein chip of a S—L—SP form wherein a substrate peptide (SP) is immobilized on a solid substrate (S) by the mediation of a linker protein (L), and a method for analyzing the interaction between a protein and its substrate peptide by such a protein chip.

### BACKGROUND ART

A protein chip is a core technology in researches to find out the function of biomolecules interacting specifically with a certain protein and to develop a method for treating and preventing diseases, which was impossible by the classic method on the basis of the data obtained from protein function analysis and network analysis.

Recent technologies on the protein chips, which have been developed till now, can be broadly classified into the following four categories:

(1) A technology of analyzing the interaction between DNA and protein on a chip by DNA microarray technology. On the chip, single-stranded oligonucleotides are converted into double-stranded oligonucleotides, and then, interacted with a restriction enzyme specific for a certain DNA sequence. Depending on whether DNA digestion occurred or not, the DNA-protein interaction is examined. Thus, this technology is useful to discover and characterize a new DNA-binding protein (Bulyk, M. L. et al., Nat. Biotechnol., 17:573-7, 1999).

(2) A technology of analyzing the reaction of antigen-antibody and the reaction of various enzymes on a protein chip, including restriction enzymes, peroxidase, phosphatase, protein kinase and the like (US 2002/0055186A1; WO 01/83827A1; Braunwalder, A. et al., Anal. Biochein., 234:23-6, 1996; Houseman, B. et al., Nat. Biotechnol., 20:270-4, 2002; and Ruud, M. et al., Nat. Biotechnol., 18:989-94, 2000). Particularly, this technology can be applied to mass searching, biochemical analysis, the analysis of new drug candidates, diagnosis of diseases and the like, by protein-protein interaction, kinase-substrate peptide interaction, and protein-ligand binding reaction. However, in a case of immobilizing a substrate peptide specific for kinase or a protein with low molecular weight, there is a limitation that the immobilized substance is buried due to a blocker BSA serving to inhibit non-specific immobilization. Furthermore, it was reported that when different kinds of antibodies were immobilized on a chip and reacted with a fluorescence labeled antigen mixture, only 60% of the antibodies showed a quantitative result, and only 23%, a qualitative result (MacBeath, G. et al., Science, 289:1760-3, 2000; and Haab, B. et al., Genome Biol., 2:research 0004, 2001).

(3) A technology of expressing and analyzing a large amount of proteins from cDNA libraries on a chip (WO 01/83827; WO 02/50260). This technology is useful for a mass search for the biochemical activity of proteins (Heng Zhu et al., Nat. Genet., 26:283-9,2000).

(4) A technology of analyzing a sample by forming a uniform and stable single layer of a biomolecule on the chip surface, maintaining the orientation of the biomolecule at a molecular level with an affinity tag (US 2002/0055125A1; U.S. Pat. No. 6,406,921; Paul, J. et al., JACS, 122:7849-50, 2000; RaVi, A. et al., Anal. Chem., 73:471-80, 2001; and Benjamin, T. et al., Trends Biotechnol., 20:279-81, 2002). For example, a protein is expressed in the form of a His-tag fusion protein and then immobilized on a chip with Ni—NTA bound chip by reaction, so that the activity of the biomolecule can be maintained. Alternatively, the protein is expressed in the form of an intein fusion protein so that it can be easily purified. Moreover, it can also be biotinylated at its certain site and immobilized in a given direction on an avidin-treated chip so that it can be maintained in a more stable and active state (Zhu et al., Science, 293:2101-5, 2001; Marie-Laure, L. et al., JACS, 124:8768-9, 2002). Furthermore, a protein (e.g., calmodulin) binding specifically to a support is expressed in a form fused with a tag (e.g., polycystein, lysine, histidine, etc.), and then immobilized on the support, so that the resulting structure is utilized for protein purification, surface plasmon resonance (SPR) analysis and fluorescence activated cell sorter (FACS) analysis (Hentz et al., Anal. Chem., 68:3939-44, 1996; Hodneland et al., PNAS, 99:5048-52, 2002; Kukar et al., Anal. Biochem., 306:504, 2002; U.S. Pat. No. 6,117,976).

However, although various protein chip technologies as described above were developed, in the current protein chip technology using a low molecular weight peptide consisting of generally less than 50 amino acids, it is difficult to induce the interaction between an immobilized peptide and a reactive protein, due to the spatial and structural problems of the macromolecular reactive protein (enzyme and antibody) interacting with the peptide. Also, it is difficult for this technology to be practically used due to many limitations in detecting the interaction using a fluorescence labeled antibody. Furthermore, this technology requires the peptide with high concentration to immobilize the peptide on the chip, so that it has reduced economic efficiency.

Thus, there has been a continuous need to develop a method capable of efficiently analyzing the interaction between the substrate peptide with low molecular weight and the reactive protein with high molecular weight on the protein chip.

Therefore, the present inventors have conducted intensive studies to develop a method capable of effectively analyzing the interaction between the reactive protein and its substrate peptide, and consequently, found that when the substrate peptide with low molecular weight is immobilized on a solid substrate by the mediation of a linker protein, and treated with the reactive protein and then the interaction between the reactive protein and the peptide is detected by an antibody, the specific interaction between the substrate peptide and the reactive protein can be analyzed in an easy and efficient manner. On the basis of these points, the present invention was perfected.

### DISCLOSURE OF THE INVENTION

Accordingly, a main object of the present invention is to provide a protein chip of a S—L—SP form wherein a substrate peptide (SP) is immobilized on a solid substrate (S) by the mediation of a linker protein (L).

Another object of the present invention is to provide a method for analyzing the interaction between a reactive protein and its substrate peptide by using such a protein chip.

The protein chip which is used to achieve the above object is produced by fusing the substrate peptide with the linker protein and immobilizing the substrate peptide on the solid substrate by the mediation of the linker protein. The substrate peptide is preferably fused with the linker protein in the form of a peptide monomer, a dimer of monomer-proline-monomer, or a multimer where monomers are linked to each other by a proline.

The fusion of the substrate peptide with the linker protein can be achieved either by culturing a microorganism transformed with a recombinant vector containing DNA coding for the substrate-linker protein and isolating the substrate-linker protein from the cultured microorganism and purifying the isolated substrate-linker protein, or by binding the substrate peptide to the linker protein chemically under laboratory conditions. However, in view of improved economic efficiency and easy production, it is preferably produced using a microbial expression system.

The substrate peptide which is used in the present invention is a substrate capable of specifically reacting with a reactive protein, and can be selected depending on the kind of the reactive protein. The linker protein which is used in the present invention is not specially limited but it is preferable to use a protein, such as leptin or malic enzyme, which can be easily expressed in a microorganism and easily purified. The solid substrate which is used in the present invention is not also specially limited but it is preferable to use a slide with aldehyde exposed which is generally used in a protein chip.

Moreover, a method for analyzing the interaction between a reactive protein and its substrate peptide using the protein chip of the present invention comprises the steps of: adding a reactive protein to the protein chip, the reactive protein showing a specific interaction with the substrate peptide immobilized on the protein chip; and detecting the interaction between the reactive protein and the substrate peptide. In this method, the reactive protein can be selected from various proteins, including enzymes and antibodies according to the purpose of analysis and it can be selected in an interdependent manner with the choice of the substrate peptide as well. For example, as the reactive protein, protein kinase A can be used, and as its substrate peptide, kemptide (SEQ ID NO: 1). Alternatively, Ab1 kinase can be used as the reactive protein, and Ab1 (SEQ ID NO: 8) as the substrate peptide.

| | |
|---|---|
| Leu Arg Arg Ala Ser Leu Gly | (SEQ ID NO: 1) |
| Glu Ala Ile Tyr Ala Ala Pro Phe Ala | (SEQ ID NO: 8) |
| Lys Lys | |

The step of detecting the interaction between the substrate peptide and the reactive protein is preferably carried out using a fluorescence labeled antibody, but various antibodies can be used for the detection, depending on the characteristics of the reactive protein. For example, when protein kinase A or Ab1 kinase is used as the reactive protein, phosphorylation of the substrate peptide by such kinases is preferably detected by using a Cy3-labeled anti-phosphorylation serine antibody or a Cy5-labeled anti-phosphorylation tyrosine antibody.

Hereinafter, the present invention will be described in detail.

When the substrate peptide reacting with an enzyme such as kinase is immobilized on the chip, and the interaction between the substrate peptide and the enzyme is detected by using an antibody, there are spatial and structural limitations in the interaction between the antibody and its substrate, and also a limitation in that the substrate peptide has insufficient stability due to its low molecular weight.

In order to solve such problems, in the present invention, the substrate peptide was expressed in E. coli in a form fused with a linker protein that is over-expressed in an insoluble aggregate form or a water-soluble form where 6 histidine residues are bound to the N-terminal region. Then, the fusion protein was immobilized on a solid substrate, thereby producing a protein chip. The stop codon of the leptin derived from human and the stop codon of a malic enzyme where 6 histidine residues are bound to the N-terminal region were removed. Then, the amino acid sequence of the substrate peptide to be fused was linked with the stop codon, so that it is expressed in a monomer form. Alternatively, two substrate peptides were linked with each other by a proline so that they are expressed in a dimer form, whereby the detection of interaction by an antibody is performed in a more efficient and easy manner.

FIG. 1 is a schematic diagram showing a leptin-kemptide, a malic enzyme-kemptide, and a leptin-Ab1 peptide, which were produced by the present invention. In FIG. 1, kemptide and Ab1 peptide as substrate peptides are fused with leptin and malic enzyme as linker proteins in a monomer form and in a dimer form where monomers are linked to each other by proline.

Concretely, in the present invention, E. coli was transformed by recombinant plasmids capable of expressing the proteins shown in FIG. 1, and then cultured, thereby giving three proteins of leptin-kemptide, malic enzyme-kemptide, and leptin-Ab1 peptide in an insoluble aggregate form or a water-soluble form. The collected proteins were purified and immobilized on an aldehyde slide to produce a protein chip. Using this protein chip, the interaction between such proteins and a fluorescence labeled antibody was analyzed. As a result, when only the substrate peptide such as the low molecular weight kemptide was immobilized on the protein chip, its interaction with the antibody did not occur, but when the peptide in a form fused with the linker protein such as leptin or malic enzyme was immobilized, its specific interaction with the antibody occurred. Also, it was found that the dimer form showed a higher reactivity than that of the monomer form.

### BRIEF DESCRIPTION OF DRAWINGS

FIG. 1 is a schematic diagram showing leptin-kemptide, malic enzyme-kemptide, and leptin-Ab1 peptide.

FIG. 2 is a schematic diagram showing recombinant plasmids pLKM and pLKD.

FIG. 3 is a schematic diagram showing recombinant plasmid pTLMK3.

FIG. 4 is a schematic diagram showing recombinant plasmids pLAM and pLAD.

FIG. 5 is a photograph showing the fluorometric analysis of the interaction between a leptin-kemptide protein and a protein kinase A on a protein chip.

FIG. 6 is a photograph showing the fluorometric analysis for the interaction between a malic emzyme-kemptide protein and a protein kinase A on a protein chip.

FIG. 7 is a photograph showing the fluorometric analysis for the interaction between a leptin-Ab1 peptide and Ab1 kinase on a protein chip.

### DETAILED DESCRIPTION OF THE INVENTION

The present invention will hereinafter be described in further detail by examples. It will however be obvious to a person skilled in the art that these examples are given for illustrative purpose only, and the scope of the present invention is not limited to or by these examples.

### EXAMPLE 1

### Construction of Recombinant Plasmid

(1) Construction of recombinant plasmids pLKM and pLKD

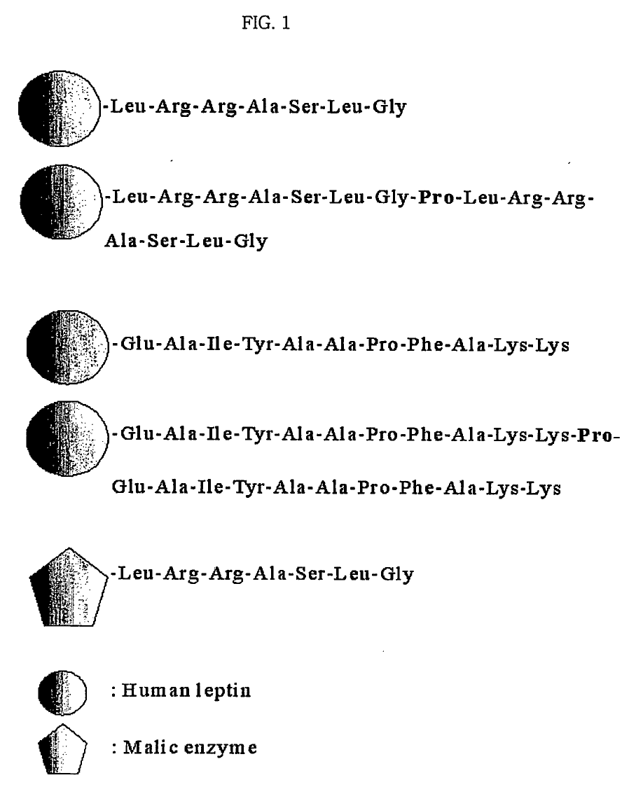

Recombinant plasmids pLKM and pLKD expressing a leptin-kemptide protein (FIG. 1) specific for a protein kinase A were constructed. To fuse a kemptide (SEQ ID NO: 1) which is a substrate peptide specific for the protein kinase A with a human leptin in a monomer form, PCR was performed using recombinant plasmid pEDOb5 (Jeong, et al., AppL Environ. Microbiol., 65(7):3027-32, 1999) containing a 414 bp leptin gene, as template DNA, and the following primer 1 containing the digestion site of restriction enzymes NdeI and BamHI, and the following primer 2 containing a kemptide gene sequence.

| | |
|---|---|
| Primer 1: | |
| (SEQ ID NO: 2) | 5′-CGGAATTCATATGGTGCCCATCCAAAA |
| | AGTCCA-3′ |
| Primer 2: | |
| (SEQ ID NO: 3) | 5′-GCGGATCCTTAGCCCAGGCTCGCACGA |
| | CGCAGGCACCCAGGGCTGAGG-3′ |

Furthermore, for fusion in a dimer form, PCR was performed using the same template DNA as above, and the primer 1 and the following primer 3, thereby obtaining template DNA from which a BamHI digestion site and a stop codon had

| | |
|---|---|
| Primer 3: | |
| (SEQ ID NO: 4) | 5′-GCGGATCCTTAGCCCAGGCTCGCGCG |
| | GCGCAGGGGGCCCAGGCTCGCACGACG-3′ |

Then, PCR using the following primer 4 was performed to obtain a DNA containing a gene coding for a protein form where a kemptide containing the digestion site of restriction enzyme BamHI and a stop codon is fused in a dimer form to the C-terminal.

| | |
|---|---|
| Primer 4: | |
| (SEQ ID NO: 5) | 5′-GCGGATCCTTAGCCCAGGCTCGCGCG |
| | GCGCAGGGGGCCCAGGCTCGCACGACG-3′ |

The PCR was performed as follows: first denaturation at 94° C. for 5 minutes; 30 cycles consisting of second denaturation at 94° C. for 1 minutes, annealing at 56° C. for 50 seconds and extension at 72° C. for 90 seconds; and final extension at 72° C. for 5 minutes. The amplified DNA resulting from the PCR was subjected to agarose gel electrophoresis to isolate about 435 bp and 459 bp DNAs. The isolated DNAs were digested with NdeI and BamHI to give DNA fragments.

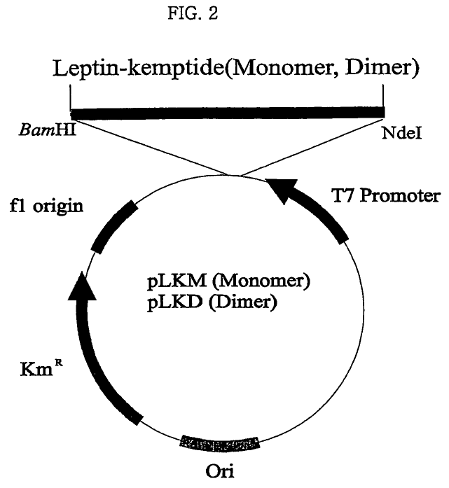

Then, plasmid pET-3a (Novagen, USA) containing a T7 promoter was digested with restriction enzymes NdeI and BamHI, mixed the DNA fragments and ligated with a T4 DNA ligase, thereby constructing recombinant plasmids pLKM and pLKD (see FIG. 2). FIG. 2 is a schematic diagram showing the recombinant plasmids pLKM and pLKD. The recombinant plasmids pLKM and pLKD contain a cDNA coding for human leptin, an oligonucleotide coding for kemptide specific for protein kinase A, and a kanamycin-resistant gene, and can express a protein of a leptin-monomer kemptide form and a protein of a leptin-dimer kemptide form, respectively.

The recombinant plasmids pLKM and pLKD were introduced into E. coli BL21(DE3) [F-ompT hsdSB(rB- mB-) gal dcm (DE3), a prophage carrying the T7 RNA polymerase gene](Novagen, USA) by a heat shock method, and cultured in an LB plate medium containing canamycin (50 μg/mL), and the transformed E. coli was screened. The recombinant plasmids pLKM and pLKD were isolated and digested with restriction enzymes NdeI and BamHI, thereby obtaining DNA fragments the size of about 435 bp and 459 bp. The DNA fragments are genes coding for a protein form where kemptide as a substrate peptide is fused with human leptin.

(2) Construction of recombinant plasmid pTLMK3

Recombinant plasmid pTLMK3 expressing a malic enzyme-kemptide protein (FIG. 1) specific for protein kinase A was constructed. Using the chromosomal DNA of E. coli W3110 (λ-, F-, prototroph) derived from E. coli K-12, as a template, and the following primer 5 (designed to contain a sequence coding for 6 histidine residues at the N-terminal end) containing the digestion sites of restriction enzymes NcoI and XbaI, and the following primer 6 (designed to contain a kemptide gene sequence at the C-terminal end), PCR was performed under the same condition as Example 1-(1), to give an malic enzyme where 6 histidine residues are linked to the N-terminal end (Hong et al., Biotechnol. Bioeng., 20, 74(2):89-95, 2001).

| | |
|---|---|
| Primer 5: | |
| (SEQ ID NO: 6) | 5′-CATGCCATGGGCATCACCATCATCAC |
| | CATGATATTCAAAAAAGAGTG-3′ |
| Primer 6: | |
| (SEQ ID NO: 7) | 5′-GCTCTAGATTAGCCCAGGCTCGCAC |
| | GACGCAGGATGGAGGTACGGCGGTA-3′ |

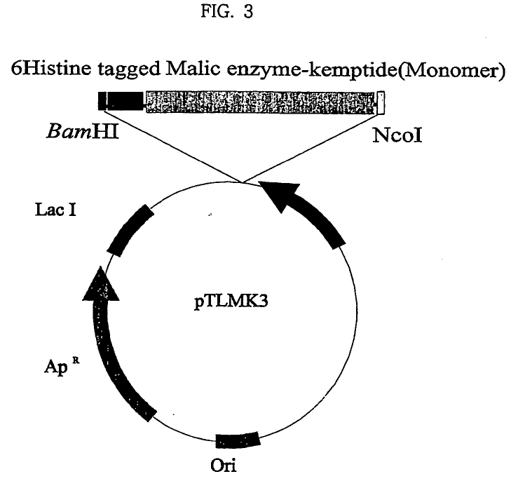

The amplified DNA resulting from the PCR was subjected to agarose gel electrophoresis to isolate DNA about the size of 1782 bp. The isolated DNA was digested with restriction enzymes NcoI and XbaI, and then inserted into plasmid pTrc99A (Pharmacia Biotech Co., Sweden) digested with the same restriction enzymes, thereby constructing recombinant plasmid pTLMK3 (FIG. 3). FIG. 3 is a schematic diagram showing the recombinant plasmid pTLMK3. This recombinant plasmid pTLMK3 contains a cDNA coding for a malic enzyme derived from E. coli, an oligopeptide coding for kemptide, and an ampicillin-resistant gene, and can express a malic enzyme-monomer kemptide protein where 6 histidine residues are linked to the N-terminal end.

E. coli XL1-Blue (Stratagene, La Jolla, USA) was transformed by the recombinant plasmid pTLMK3 and cultured in an LB plate medium containing ampicillin (50μg/mL). The transformed E. coli was screened and the recombinant plasmid pTLMK3 was isolated from the E. coli.

(3) Construction of recombinant plasmids pLAM and pLAD

Recombinant plasmids pLAM and pLAD expressing the leptin-Ab1 peptide (FIG. 1) specific for an Ab1 kinase were constructed. A DNA sequence coding for Ab1 (SEQ ID NO: 8) was digested with restriction enzymes NdeI and BamHI to give DNA fragments about the size of 477 bp and 516 bp. Meanwhile, plasmid pET-30a (Novagen, USA) containing a T7 promoter was digested with the same restriction enzymes NdeI and BamHI.

To give a 438 bp human leptin gene selected as a linker protein, PCR was performed using recombinant plasmid pEDOb5 (Jeong et al., Appl. Environ. Microbiol., 65:3027-32, 1999) as a template, and the following primers 7 and 8 containing the digestion sites of restriction enzymes NdeI and BamHI.

Primer 7:
(SEQ ID NO: 9)
5′-CGGAATTCATATGGTGCCCATCCAAAA
AGTCCA-3′
Primer 8:
(SEQ ID NO: 10)
5′-CGGGATCCTCATTATTTTTTTTTCGCA
AACGGCGCCGCATAGATCGCTTCGCACCCAGGGCTGAGGT-3′

Furthermore, for fusion in a dimer form, PCR was performed using the same template DNA as above, the primer 1 and the following primer 9, to obtain template DNA from which the digestion site of BamHI and a stop codon had been deleted.

Primer 9:
(SEQ ID NO: 11)
5′-CGGGATCCTTTTTTTTTCGCAAACGG
CGCCGCATAGATCGCTTCGCACCGAGGGCTGAGGT-3

The template was amplified by PCR using the synthesized primers 7 and 9, and the following primer 10 containing the digestion site of BamHI was constructed to obtain a dimer PCR product.

Primer 10:
(SEQ ID NO: 12)
5′-CGGGATCCTCATTATTTTTTTTTCGC
AAACGGCGCCGCATAGATCGCGGGTTTTTTTTTCGCAAACGGCG
C-3′

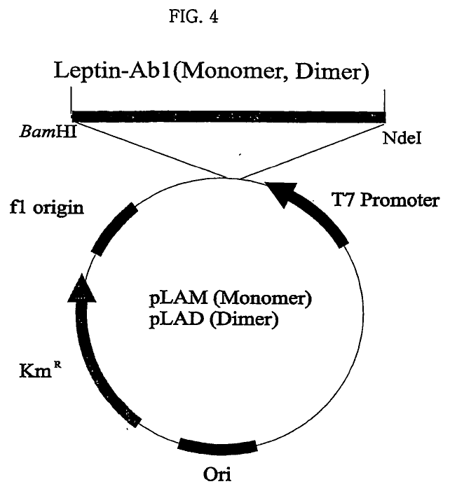

The amplified DNA resulting from the PCR was subjected to an agarose gel electrophoresis to isolate DNA fragments about the size of 477 bp and 516 bp. The isolated DNAs were digested with restriction enzymes NdeI and BamHI, and then inserted into plasmid pET-30a digested with the same restriction enzymes, thereby constructing recombinant plasmids pLAM and pLAD (FIG. 4). FIG. 4 is a schematic diagram showing the recombinant plasmids pLAM and pLAD. The plasmids pLAM and pLAD contains a cDNA coding for human leptin, an oligonucleotide coding for Ab1, and a kanamycin-resistant gene, and can express a protein of a leptin monomer-Ab1 form and a protein of a leptin dimer-Ab1 form, respectively.

E. coli BL21(DE3) was transformed with the recombinant plasmids pLAM and pLAD, and cultured in an LB plate medium containing kanamycin (50 μg/mL). After screening the transformed E. coli, recombinant plasmids pLAM and pLAD were isolated from the transformed E. coli.

### EXAMPLE 2

### Analysis of Interaction Between Leptin-kemptide Protein and Protein Kinase A Using Protein Chip on which Leptin-kemptide Protein was Immobilized

(1) Preparation of protein chip on which leptin-kemptide protein was immobilized

The recombinant E. coli transformed with the recombinant plasmids pLKM and pLKD containing a gene coding for a leptin-kemptide protein was inoculated into 200 mL of an LB medium and cultured at 37° C. When the optical density at a 600 nm wavelength reached 0.7, 1 mM IPTG was added to induce the expression of the leptin-kemptide protein. After 4 hours, the culture broth was centrifuged at 4° C. and 6,000 rpm for 5 minutes, and the resulting precipitate was washed with 100 mL of TE buffer (Tris-HCl 10 mM; EDTA 1 mM, pH 8.0). The washed substance was centrifuged at 4° C. and 6,000 rpm for 5 minutes, and then suspended in 100 mL of TE buffer. The resulting cell was disrupted in an ultrasonicator (Branson Ultrasonics Co., USA).

The disrupted solution was centrifuged at 4° C. and 6,000 rpm for 30 minutes, and the resulting particulate was suspended in 10 mL of a denaturation solution (8 M urea, 10 mM Tris, pH 8.0). The suspension was stirred for 4 hours at room temperature and dissolved, and then the stirred solution was centrifuged at 4° C. at 6000 rpm for 30 minutes. The supernatant was collected and filtered through a 0.2 μm filter. Protein contained in the filtrate was quantified by the Bradford protein assay (Bradford, M. M., Anal. Biochem., 72:248-54, 1976), and then, diluted with a fixation solution (40% glycerol, PBS, pH 7.4) to the concentration of 1 mg/mL.

The diluted solution was spotted on an aldehyde slide at intervals of 300-500 μm (500/cm²) using a microarrayer (Yoon, S. H. et al, J., Microbiol. Biotechnol., 10:21-6, 2000), and immobilized in a 30° C. humid chamber for 1 hour. Then, it was reacted with a blocking solution (1% BSA, PBS, pH 7.4) at room temperature for 1 hour, thereby producing a protein chip. For use as a control group, 1 mg/mL kemptide, 1 mg/mL bovine serum albumin (BSA), 1 mg/mL leptin and phosphate buffer were diluted with the same fixation solution.

(2) Analysis of interaction between leptin-kemptide protein and protein Kinase A

The protein chip produced in Example 2-(1) was washed three times with washing solution (20 mM Tris, 150 mM NaCl, 10 mM EDTA, 1 mM EGTA, 0.1% Triton-X100, pH 7.5) for 5 minutes, and then washed with kinase solution (50 mM Tris, 10 mM MgCl₂, pH 7.5). Then, 200 (1 kinase solution containing 100 (1 ATP was spread on the chip, covered with a cover well and then subjected to interaction with the leptin-kemptide protein for 1 hour.

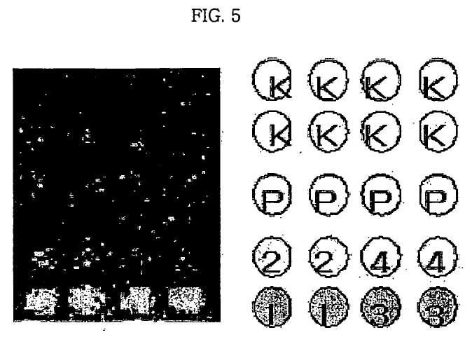

After the interaction, the protein chip was sufficiently washed with kinase solution, and 200(1 kinase reactive solution (containing 100 (M ATP and 10 units of cAMP-dependent protein kinase) was spread on the chip, covered with a cover well and then subjected to interaction with the leptin- kemptide protein for 1 hour. After the interaction, the protein chip was sufficiently washed with phosphate buffer (PBS, pH 7.4), and then the leptin-kemptide protein on the chip was subjected to interaction with a Cy3-labeled anti-phosphorylation serine antibody. Then, the resulting solution was sufficiently washed, centrifuged at 200 g for 1 minute to completely remove excess solution. Next, the reaction was analyzed using ScanArray 5000 (Axon Instrument, Forster, USA) laser scanner (FIG. 5).

FIG. 5 is a photograph showing the fluorometric analysis of the interaction between the leptin-kemptide protein and the protein kinase A. In FIG. 5, 1 represents 1 mg/mL leptin-dimer kemptide, 2 represents 10-fold diluted reptin dimer, 3 represents 1 mg/mL leptin-monomer kemptide, 4 represents 10-fold diluted leptin monomer, P represents PBS, and K represents kemptide (1 mg/mL).

As shown in FIG. 5, the protein kinase A showed a specific interaction with the kemptide of a form fused with leptin, but had no interaction with the kemptide of a single form. In a diluted state as shown by 2 and 4, the dimer showed a higher reactivity than the monomer. Thus, as expected, it could be found that the low molecular weight peptide had low reactivity with the enzyme protein, but the peptide of a form fused with the linker protein like leptin had high reactivity, and the peptide form, which had been fused with the linker protein in a dimer protein, showed a higher reactivity than the peptide form that had been fused in a monomer form.

### EXAMPLE 3

### Analysis of Interaction Between Malic Enzyme-kemptide Protein and Protein Kinase A Using Protein Chip on which Malic Enzyme-kemptide Protein was Immobilized

(1) Preparation of protein chip on which malic enzyme-kemptide protein was immobilized

The recombinant E. coli transformed with the recombinant plasmid pTLMK3 containing the gene coding for the malic enzyme-kemptide protein was cultured in the same manner as in Example 2-(1), and then the cultured cells were disrupted by an ultrasonicator. The disrupted solution was centrifuged at 4° C. and 6,000 rpm for 30 minutes, and the supernatant was collected, dialyzed by equilibrium solution (50 mM NaH₂PO_{4,} 300 mM NaCl, 10 mM imidazole, pH 8.0), purified by nickel-chelate resin (Quiagen, USA), dialyzed by PBS, and then filtered through a 0.2μm filter. Protein contained in the filtrate was quantified in the same manner as in Example 2-(1), diluted and then spotted on an aldehyde slide, thereby producing a protein chip.

(2) Analysis of interaction between malic enzyme-kemptide protein and protein kinase A

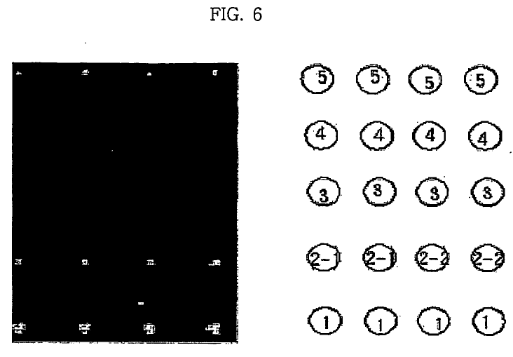

Using the protein chip produced in Example 3-(1 ), the interaction between the malic enzyme-kemptide protein and the protein kinase A was analyzed in the same manner as Example 2-(2) (see FIG. 6). FIG. 6 is a photograph showing the fluorometric analysis of the interaction between the malic enzyme-kemptide protein and the protein kinase A. In FIG. 6, 1 represents a positive control (Cy3-labeled anti-phosphorylation serine antibody), 2-1 represents a leptin-monomer kemptide, 2-2 represents a leptin-dimer kemptide, 3 represents a kemptide, 4 represents PBS, and 5 represents a malic enzyme-monomer kemptide.

As shown in FIG. 6, the protein kinase A showed a specific interaction with the kemptide of a form fused with the leptin or malic enzyme, but had little or no interaction with the kemptide of a single form. Thus, like the results shown in FIG. 5, it could be found that the low molecular weight peptide on the protein chip showed low reactivity with the enzyme protein, but the peptide of a form fused with the linker protein such as malic enzyme or leptin had high reactivity with the enzyme protein.

### EXAMPLE 4

### Analysis of Interaction Between Leptin-Ab1 Peptide and Ab1 Kinase Using Protein Chip on which Leptin-Ab1 Peptide was Immobilized

(1) Preparation of protein chip on which leptin-Ab1 peptide was immobilized E. coli was transformed with each of recombinant plasmids pLAM and pLAD containing a gene coding for a leptin-Ab1 peptide, and cultured in the same manner as Example 2-(1). The leptin-Ab1 peptide was isolated from the cultured E. coli and spotted on an aldehyde slide, thereby producing a protein chip.

(2) Analysis of interaction between leptin-Ab1 peptide and Ab1 kinase

The protein chip produced by Example 4-(1) was washed three times with washing solution (PBS, pH 7.5) for 5 minutes, and washed with kinase solution (50 mM Tris, 10 mM MgCl_{2,} 1 mM EGTA, 2 mM dithiothreitol, 0.01% Brij 36, pH 7.5). Then, 200 μl kinase solution containing 100 μM ATP was spread on the chip, covered with a cover well and subjected to interaction with the leptin-Ab1 peptide for 1 hour. When the interaction was finished, the protein chip was sufficiently washed with kinase solution, and then, 200 μl kinase solution (containing 100 μM ATP and 100 units of Ab1 kinase) was spread on the chip, covered with a cover well, and subjected to interaction with the leptin-Ab1 peptide for 1 hour.

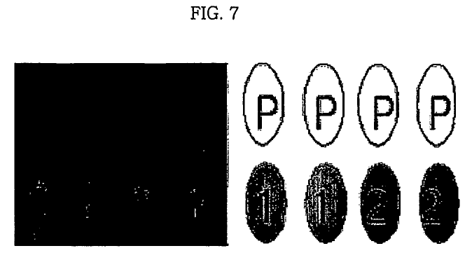

After the interaction, the protein chip was sufficiently washed with phosphate buffer (PBS, pH 7.4), and then, the solution on the chip was subjected to interaction with a Cy5-labeled anti-phosphorylation tyrosine antibody, sufficiently washed, and centrifuged at 200 g for 1 minute to remove excess solution completely. Then, the result of the interaction was analyzed (FIG. 7).

FIG. 7 is a photograph showing the fluorometric analysis of the interaction between the leptin-Ab1 peptide and the Ab1 kinase. In FIG. 7, 1 represents a leptin-dimer Ab1, 2 represents a leptin-monomer Ab1, and P represents PBS. As shown in FIG. 7, it could be found that the Ab1 kinase showed a specific interaction with the Ab1 monomer and dimer fused with leptin.

While the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

Industrial Applicability

As described and demonstrated above, the use of the inventive protein chip of a S—L—SP form allows an increase in the reactivity between peptide with the low molecular weight and enzyme with the high molecular weight and between the peptide and the reactive antibody on the protein chip, so that the interaction between the peptide and the protein can be analyzed in a rapid and effective manner. Thus, the protein chip according to the present invention will be advantageously used in a efficient and economic manner for studies and applications, including mass-searching, biochemical analysis, the analysis of new drug candidates, the diagnosis of diseases, and the like.

1. A protein chip of a S—L—SP form wherein a substrate peptide (SP) is immobilized on a solid substrate (S) by the mediation of a linker protein (L).

2. The protein chip according to claim 1, wherein the linker protein is comprises leptin or malic enzyme.

3. The protein chip according to claim 1, wherein the substrate peptide is fused with the linker protein in the form of a peptide monomer, a dimer of monomer-proline-monomer, or a multimer where monomers are linked to each other by a proline.

4. The protein chip according to claim 3, wherein the peptide monomer is comprises kemptide (SEQ ID NO: 1) or Ab1 (SEQ ID NO: 8).

5. The protein chip according to claim 1, wherein the solid substrate is comprises a side with exposed aldehyde.

6. A method for analyzing the interaction between a reactive protein and its substrate peptide using the protein chip of claim 1, comprising the steps of:
(a) adding a reactive protein to the protein chip, the reactive protein showing a specific interaction with the substrate peptide immobilized on the protein chip; and
(b) detecting the interaction between the reactive protein and the substrate peptide.

7. The method according to claim 6, wherein the reactive protein is comprises an enzyme or an antibody.

8. The method according to claim 7, wherein the enzyme is comprises protein kinase A or Ab1 kinase.

9. The method according to claim 6, wherein the step of detecting the interaction between the substrate peptide and the reactive protein is carried out by using a fluorescence labeled antibody.

10. The method according to claim 8, wherein the step of detecting a phosphorylation of the substrate peptide by kinase is carried out by using a Cy3-labeled anti-phosphorylation serine antibody or a Cy5-labeled anti-phosphorylation tyrosine antibody.
